SEQUENCE LISTING (1 sequence)

SEQ ID NO: 1 - DNA, 7916 bp, Artificial Sequence
  Nucleotide sequence encoding viral genome of recombinant lentivirus.
aagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggagaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcagccgctgatcttcagacctggaggaggagatatgagggactattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgaccacgagactagcatcgagaagtatatgccttgatttcagaatcttgccaactagattcagaatttgactgggagagaggacaaggaccacttgagactcatattttatttccagaatctagcatctacctacctaggtattgaataagaaaaatgaagttaaggtggttgatggtaacactatgctaataactgcagagccagaagcaccataagggacatgataagggagccagcagacctctgatctcttcctgaatgctaatcttaaacatcctgaggaagaatgggacttccatttggggtgggcctatgacagggtaataagacagtagtgaatatcaagctacaaaaagccgcctttcaaattcttctcagtcctaacttttcatactaagcccagtccttccaaagcagactgtgaaagagtgatagttccgggagactagcactgcagattccgggtcactgtgagtgggggaggcagggaagaagggctcacaggacagtcaaaccatgacccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcatatgcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcagcggccgcttgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagagatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtataccctgttacttatccccttcctatgacatgaacttaaccatagaaaagaaggggaaagaaaacatcaagcgtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctcctcaggagtcagatgcaccatggctcgagtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagctctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcgtccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagccattctccccacatagatcatctcagcagggttcaggaagataaaggaggatcaaggtcgaaggtaggaactaaggaagaacactgggcaagtggatccaggttgtccaacgctgaaagtaggaatctaagcactagtctctggatgctaggagggcctctgcatgggtaactctttcaactagccaggggctggactgtggagaaaccatttccagatagaagtgaggagattccagcaggaaacacttaagagaggatccctggaagttcggggcaggaggctccctgtcacatgaaggaaacctgctcagcgtaggctctaggttcttccctactcttatccaatggggcttctgattttaagccagtcttcacaaagccccagatttctacatgctggagccctgaatgggcaggaagcattgcggtttccatttcattcagagctctttcataccctgcttccccatagtttgtctcaacatttctgttgataatctgattcatgcaaccaagaattatgagagagccttcagtcatgcctaggcctgcatttattgttgtgcatatgatggggtagcagacaagaaaacgttaacagcatagactttctcagagctatatgagtttgggaattaatcaagaccagccttttgctcagagctcatgcatttaataaacggaaattttgtattgaaatgagagccattggaaatcatttactccagactcctacttataaaaagagaaactgaggctcagagaagggtggggactttctcagtatgacatggaaatgatcaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattctcacactaagaaaaagaatgagatgtctacatataccctgcgtcccctcttgtgtactggggtccccaagagctctctaaaagtgatggcaaagtcattgcgctagatgccatcccatctattataaacctgcatttgtctccacacaccagtcatggacaataaccctcctcccaggtccacctggacttcgagctcggtacgtttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttac

(19) United States
(12) Patent Application Publication
(10) Pub. No.: US 2024/0350664 A1
(43) Pub. Date: Oct. 24, 2024

(54) GENE THERAPY BASED ADMINISTRATION OF LENTIVIRUS VECTOR FOR TREATING HEMOGLOBINOPATHIES

(71) Applicant: INTAS PHARMACEUTICALS LTD., Thaltej, Ahmedabad, Gujarat (IN)

(72) Inventors: Ajit GUPTA, Sanand Ahmedabad, Gujarat (IN); Lakshmikanth GANDIKOTA, Sanand Ahmedabad, Gujarat (IN)

(21) Appl. No.: 18/252,153

(22) PCT Filed: Nov. 19, 2021
(86) PCT No.: PCT/IB2021/060740

§ 371 Date: May 8, 2023

(30) Foreign Application Priority Data
Nov. 23, 2020 (IN) 202021050878

(51) Int. Cl. A61K 48/00; A61K 38/42; A61P 7/06; C12N 15/86

(52) U.S. Cl. CPC A61K 48/005; A61K 38/42; A61P 7/06; C12N 15/86; C12N 2740/15043; C12N 2740/15071; C12N 2830/001; C12N 2830/48; C12N 2830/50

(57) ABSTRACT

The invention provides persistent expression of β-globin gene by using cell and/or gene therapy based administration of nucleotide sequence encoding β-globin gene to treat thalassemia and sickle cell anemia. Lentivirus (LV) based viral vector system containing an expression cassette of β-globin gene. Whereas, the lentivirus particle is packed with genes expressing functional β-globin gene in erythroid cell lineage specifically.

### RELATED APPLICATION

This application is related to Indian Provisional Application 202021050878 filed on 23 Nov. 2020 and is incorporated herein in its entirety.

### FIELD OF THE INVENTION

The present invention relates to lentivirus (LV) vector comprising gene encoding β-globin gene, composition thereof and method of using the same.

### BACKGROUND OF THE INVENTION

Beta (β)-thalassemia is genetic disorders resulting in the synthesis of little or no β-globin chains leading to α/β globin chain imbalance. Management of this disease and the complications of its treatment (e.g. life-long red blood cell transfusion, iron chelation and splenectomy etc.), impose high costs on healthcare systems. Patients with β-thalassemia major require regular transfusions of red blood cells (RBCs) to survive. However, repeated transfusions cause iron overload, with life-threatening complications, such as endocrine dysfunction, cardiomyopathy, liver disease and ultimately, premature death. In the absence of transfusion, patients with β-thalassemia major die within the first five years of life, and even with transfusions, only 50-65% of patients live beyond the age of 35 years in high-income countries.

The approaches to manage the disease now evolved beyond the blood transfusion e.g. fetal globin gene reactivation by pharmacological compounds injected into patients throughout their lives, allogeneic hematopoietic stem cell transplantation (HSCT) and cell/gene therapy.

HSCT is currently the only treatment shown to provide an effective, definitive cure for β-thalassemia. However, this procedure remains risky and histocompatible donors are identified for only a small fraction of patients. In addition, HSC transplants are also associated with significant mortality and morbidity in subjects that have SCD or severe thalassemia. The significant mortality and morbidity is due in part to pre-HSCT transfusion-related iron overload, graft-versus-host disease (GVHD), and high doses of chemotherapy/radiation required for pre-transplant conditioning of the subject, among others.

New pharmacological compounds are being tested, but none has yet made it into common clinical practice for the treatment of β-thalassemia major. Gene therapy is in the experimental phase. It is emerging as a powerful approach without the immunological complications of HSCT, but with other possible drawbacks. Rapid progress is being made in this field, and long-term efficacy and safety studies are underway.

Sickle cell disease (SCD) is a pathogenic hemoglobin (sickle hemoglobin) disorder originated due to a point mutation in the β-globin gene that causes hemoglobin polymerization, sickling of RBCs, hemolytic anemia, vaso-occlusion, end-organ damage, and early mortality. Hydroxyurea (HU) and L-glutamine are Food and Drug Administration (FDA)-approved widely accepted treatment for SCD that induces fetal hemoglobin expression and thereby reduces sickling. However, moderate health benefits, frequent dosing and variable response among patients remain concerns.

These limitations have prompted the development of gene base β-globin for sickle cell anemia and thalassemia.

### OBJECTS OF THE INVENTION

The principal object of the present invention is to provide lentivirus (LV) vector comprising nucleotide sequence encoding β-globin gene.

Another object of the present invention is to provide lentivirus (LV) vector comprising nucleotide sequence encoding β-globin gene, wherein said β-globin gene comprises threonine to glutamine substitution at amino acid position 88 (T88Q).

Another object of the present invention is to provide lentivirus (LV) vector comprising nucleotide sequence having SEQ ID NO: 01 encoding β-globin gene (T88Q).

Another object of the present invention is to provide viral genome of recombinant lentivirus (LV) particle containing β-globin gene (T88Q), wherein said viral genome comprises:
(a) One or more left and right long terminal repeat (LTR) sequences in its native or truncated form, that flank the 5′ or 3′ terminus of the heterologous polynucleotide sequence;
(b) HIV1 psi;
(c) RRE (Rev response element of HIV-1);
(d) An expression control elements, wherein expression control elements comprises human β-globin promoter, a human β-globin LCR and a human β-globin enhancer sequence; and
(e) SV40 PolyA Signal.

Another object of the present invention is to provide viral genome of recombinant lentivirus (LV) particle encoded by nucleotide sequence of SEQ ID NO: 01 comprising:
(a) One or more left and right long terminal repeat (LTR) sequences in its native or truncated form, that flank the 5′ or 3′ terminus of the heterologous polynucleotide sequence;
(b) HIV1 psi;
(c) RRE (Rev response element of HIV-1);
(d) An expression control elements, wherein expression control elements comprises human β-globin promoter, a human β-globin LCR and a human β-globin enhancer sequence; and
(e) SV40 PolyA Signal.

Another object of the present invention is to provide recombinant lentivirus (LV) vector, wherein said viral vector comprising:
(a) RSV Promoter;
(b) 5′ LTR;
(c) HIV-1 psi;
(d) RRE;
(e) cPPT/CTS;
(f) 3′ Beta Enhancer;
(g) HBB;
(h) Beta Globin Locus control Region (HS2-HS3-HS3.2-HS4);
(i) 3′ LTR (Delta-U3);
(j) SV40 poly (A) signal; and
(k) SV40 Ori.

Another object of the present invention is to provide recombinant lentivirus (LV) vector, wherein said viral vector encoded by nucleotide sequence of SEQ ID NO: 01 comprising:
(a) RSV Promoter;
(b) 5′ LTR;
(c) HIV-1 psi;
(d) RRE;
(e) cPPT/CTS;
(f) 3′ Beta Enhancer;
(g) HBB;
(h) Beta Globin Locus control Region (HS2-HS3-HS3.2-HS4);
(i) 3′ LTR (Delta-U3);
(j) SV40 poly (A) signal; and
(k) SV40 Ori.

Another object of the present invention is to provide persistent expression of β-globin gene by using cell/gene therapy based administration of nucleotide sequence encoding β-globin gene for the treatment of thalassemia and sickle cell anemia.

Another object of the present invention is to provide therapies to treat thalassemia using hematopoietic progenitor cells or mammalian stem cells transduced with lentivirus vector containing nucleic acid transgene encoding functional β-globin gene.

Another object of the present invention is to provide methods for recombinant lentivirus (LV) particles preparation wherein, the lentivirus (LV) particle is packed with genes expressing functional β-globin gene in erythroid cell lineage specifically.

### SUMMARY OF THE INVENTION

The principal aspect of the present invention is to provide lentivirus (LV) vector comprising nucleotide sequence encoding β-globin gene.

Another aspect of the present invention is to provide lentivirus (LV) vector comprising nucleotide sequence encoding β-globin gene wherein, said β-globin gene comprises threonine to glutamine substitution at amino acid position 88 (T88Q).

Another aspect of the present invention is to provide lentivirus (LV) vector comprising nucleotide sequence having SEQ ID NO: 01 encoding β-globin gene (T88Q).

Another aspect the present invention is to provide viral genome of recombinant lentivirus (LV) particle containing β-globin gene (T88Q), wherein said viral genome comprises:
(a) One or more left and right long terminal repeat (LTR) sequences in its native or truncated form, that flank the 5′ or 3′ terminus of the heterologous polynucleotide sequence;
(b) HIV1 psi;
(c) RRE (Rev response element of HIV-1);
(d) An expression control elements, wherein expression control elements comprises human β-globin promoter, a human β-globin LCR and a human β-globin enhancer sequence; and
(e) SV40 PolyA Signal.

Another object of the present invention is to provide viral genome of recombinant lentivirus (LV) particle encoded by nucleotide sequence of SEQ ID NO: 01 comprising:
(a) One or more left and right long terminal repeat (LTR) sequences in its native or truncated form, that flank the 5′ or 3′ terminus of the heterologous polynucleotide sequence;
(b) HIV1 psi;
(c) RRE (Rev response element of HIV-1);
(d) An expression control elements, wherein expression control elements comprises human β-globin promoter, a human β-globin LCR and a human β-globin enhancer sequence; and
(e) SV40 PolyA Signal.

Another aspect of the present invention is to provide recombinant lentivirus (LV) vector, wherein said viral vector comprising:
(a) RSV Promoter;
(b) 5′ LTR;
(c) HIV-1 psi;
(d) RRE;
(e) cPPT/CTS;
(f) 3′ Beta Enhancer;
(g) HBB;
(h) Beta Globin Locus control Region (HS2-HS3-HS3.2-HS4)
(i) 3′ LTR (Delta-U3);
(j) SV40 poly (A) signal; and
(k) SV40 Ori.

Another object of the present invention is to provide recombinant lentivirus (LV) vector, wherein said viral vector encoded by nucleotide sequence of SEQ ID NO: 01 comprising:
(a) RSV Promoter;
(b) 5′ LTR;
(c) HIV-1 psi;
(d) RRE;
(e) cPPT/CTS;
(f) 3′ Beta Enhancer;
(g) HBB;
(h) Beta Globin Locus control Region (HS2-HS3-HS3.2-HS4)
(i) 3′ LTR (Delta-U3);
(j) SV40 poly (A) signal; and
(k) SV40 Ori.

Another aspect of the present invention is to provide persistent expression of β-globin gene by using cell/gene therapy based administration of nucleotide sequence encoding β-globin gene for the treatment of thalassemia and sickle cell anemia.

Another aspect of the present invention is to provide therapies to treat thalassemia using hematopoietic progenitor cells or mammalian stem cells transduced with lentivirus vector containing nucleic acid transgene encoding functional β-globin gene.

Another aspect of the present invention is to provide methods for recombinant lentivirus (LV) particles preparation wherein, the lentivirus (LV) particle is packed with genes expressing functional β-globin or its variant in erythroid cell lineage specifically.

### BRIEF DESCRIPTION OF DRAWINGS

In order that the disclosure may be readily understood and put into practical effect, reference will now be made to exemplary embodiments as illustrated with reference to the accompanying figures. The figure together with detailed description below, are incorporated in and form part of the specification, and serve to further illustrate the embodiments and explain various principles and advantages, in accordance with the present disclosure wherein:

FIG. 1: Schematic representation of vector map (pGT B Thal T88Q) with transgene and regulatory elements.

FIG. 2: Schematic representation of lentivirus vector gene expression cassette (pGT B Thal T88Q).

### DETAILED DESCRIPTION OF THE INVENTION

The present invention provides improved compositions and methods for gene therapy, particularly in the treatment of hemoglobinopathies. In one embodiment, improved lentiviral vectors are used to deliver therapeutic gene products to hematopoietic stem cells, including, but not limited to, erythroid progenitors.

The lentiviral vector are designed to express a β-globin gene in mammalian, and more particularly, human cells. These β-globin genes are well suited for treatment of thalassemia and sickle cell anemia due to mutations in the β-globin gene.

Lentivirus based viral vector system containing an expression cassette of β-globin gene that is expressed in erythroid cell lineage specifically. Such vectors can be used for the treatment of thalassemia and sickle cell anemia by using gene therapy based administration of nucleotide sequence encoding β-globin gene.

### Definition

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., lentiviral vectors.

A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s).

The term “viral vector” may refer either to a virus or viral particle capable of transferring a nucleic acid into a cell or to the transferred nucleic acid itself. Viral vectors and transfer plasmids contain structural and/or functional genetic elements that are primarily derived from a virus.

The terms “lentiviral vector” and “lentiviral expression vector” may be used to refer to lentiviral transfer plasmids and/or infectious lentiviral particles in particular embodiments. Where reference is made herein to elements such as cloning sites, promoters, regulatory elements, heterologous nucleic acids, etc., it is to be understood that the sequences of these elements are present in RNA form in the lentiviral particles contemplated herein and are present in DNA form in the DNA plasmids contemplated herein.

The terms “recombinant cell” or “chimeric recombinant cell” may be used to refer to a manipulated natural cell population or a cell line in particular embodiments. Where reference is made herein to any cell stably expressing β-globin.

The terms “cell therapy” or “gene therapy” should be inclusively interchangeable and may be used to refer to inclusive ex vivo or/or in vivo therapy in particular embodiments. Where reference is made herein to therapeutic application of lentivirus and/or any in vitro, ex vivo or in vivo cell stably expressing β-globin.

The term “long terminal repeat (LTR)” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R and U5. The U3 region contains the enhancer and promoter elements. The U5 region is the sequence between the primer binding site and the R region and contains the polyadenylation sequence. The R (repeat) region is flanked by the U3 and U5 regions. The LTR composed of U3, R and U5 regions and appears at both the 5′ and 3′ ends of the viral genome. Adjacent to the 5′ LTR are sequences necessary for reverse transcription of the genome and for efficient packaging of viral RNA into particles (the Psi site). Proviral inserts comprise two copies of the 3′ viral LTR, one copy that replaces the 5′ viral LTR and the 3′ viral LTR.

The term “poly (A) site” or “poly (A) sequence” as used herein denotes a DNA sequence which directs both the termination and polyadenylation of the nascent RNA transcript by RNA polymerase II. Polyadenylation sequences can promote mRNA stability by addition of a poly(A) tail to the 3′ end of the coding sequence and thus, contribute to increased translational efficiency. Cleavage and polyadenylation is directed by a poly (A) sequence in the RNA. The core poly (A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly (A) sequence is a synthetic poly (A) sequence (e.g., AATAAA, ATTAAA, AGTAAA). Illustrative examples of poly (A) sequences include, but are not limited to an SV40 poly (A) sequence, a bovine growth hormone poly (A) sequence, a rabbit β-globin poly(A) sequence (rPgpA), or another suitable heterologous or endogenous poly (A) sequence known in the art.

As used herein, the term “packaging signal” or “packaging sequence” refers to sequences located within the retroviral genome which are required for insertion of the viral RNA into the viral capsid or particle. Several retroviral vectors use the minimal packaging signal (also referred to as the psi [Ψ] sequence) needed for encapsidation of the viral genome. Thus, as used herein, the terms “packaging sequence,” “packaging signal,” “psi” and the symbol “Ψ,” are used in reference to the non-coding sequence required for encapsidation of retroviral RNA strands during viral particle formation.

The term “globin” as used herein refers to proteins or protein subunits that are capable of covalently or non-covalently binding a heme moiety, and can therefore transport or store oxygen. Subunits of vertebrate and invertebrate hemoglobin's, vertebrate and invertebrate myoglobin's or mutants thereof are included by the term globin. The term excludes hemocyanins. Examples of globins include α-globin or variant thereof, β-globin or variant thereof, a γ-globin or a variant thereof, and δ-globin or a variant thereof.

The term “promoter” as used herein refers to an expression control sequence that comprises a recognition site of a polynucleotide (DNA or RNA) to which an RNA polymerase binds.

The term “enhancer” refers to an expression control sequence that comprises a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “hematopoietic stem cell” or “HSC” refers to multipotent stem cells that give rise to all the blood cell types of an organism, including myeloid (e.g., monocytes and macrophages, neutrophils, basophils, eosinophil's, erythrocytes, megakaryocytes/platelets, dendritic cells), and lymphoid lineages (e.g., T-cells, B-cells, NK-cells), and others known in the art. When transplanted into lethally irradiated animals or humans, hematopoietic stem and progenitor cells can repopulate the erythroid, neutrophil-macrophage, megakaryocyte and lymphoid hematopoietic cell pool.

In the following description, certain specific details are set forth in order to provide a thorough understanding of various illustrative embodiments of the invention contemplated herein. However, one skilled in the art will understand that particular illustrative embodiments may be practiced without these details.

The main embodiment of the present invention is to provide lentivirus (LV) vector comprising nucleotide sequence encoding β-globin gene.

Another embodiment of the present invention is to provide lentivirus (LV) vector comprising nucleotide sequence encoding β-globin gene, wherein said β-globin gene comprises threonine to glutamine substitution at amino acid position 88 (T88Q).

Another embodiment of the present invention is to provide lentivirus (LV) vector comprising nucleotide sequence having SEQ ID NO: 01 encoding β-globin gene (T88Q).

Another embodiment of the present invention is to provide viral genome of recombinant lentivirus (LV) particle containing β-globin gene (T88Q), wherein said viral genome comprises:
(a) One or more left and right long terminal repeat (LTR) sequences in its native or truncated form, that flank the 5′ or 3′ terminus of the heterologous polynucleotide sequence;
(b) HIV1 psi;
(c) RRE (Rev response element of HIV-1);
(d) An expression control elements, wherein expression control elements consist of human β-globin promoter; a human β-globin LCR; and a human β-globin enhancer sequence; and
(e) SV40 PolyA Signal.

Another object of the present invention is to provide viral genome of recombinant lentivirus (LV) particle encoded by nucleotide sequence of SEQ ID NO: 01 comprising:
(a) One or more left and right long terminal repeat (LTR) sequences in its native or truncated form, that flank the 5′ or 3′ terminus of the heterologous polynucleotide sequence;
(b) HIV1 psi;
(c) RRE (Rev response element of HIV-1);
(d) An expression control elements, wherein expression control elements comprises human β-globin promoter, a human β-globin LCR and a human β-globin enhancer sequence; and
(e) SV40 PolyA Signal.

Another object of the present invention is to provide viral genome of recombinant lentivirus (LV) particle encoded by nucleotide sequence of SEQ ID NO: 01 comprising:
(a) One or more left and right long terminal repeat (LTR) sequences in its native or truncated form, that flank the 5′ or 3′ terminus of the heterologous polynucleotide sequence;
(b) HIV1 psi;
(c) RRE (Rev response element of HIV-1);
(d) HIV cPPT/CTS;
(e) An expression control elements, wherein expression control elements comprises human β-globin promoter, a human β-globin LCR and a human β-globin enhancer sequence; and
(f) SV40 PolyA Signal.

Another embodiment of the present invention is to provide recombinant Lentivirus (LV) vector, wherein said viral vector comprising:
(a) RSV Promoter;
(b) 5′ LTR;
(c) HIV-1 psi;
(d) RRE;
(e) cPPT/CTS;
(f) 3′ Beta Enhancer;
(g) HBB;
(h) Beta Globin Locus control Region (HS2-HS3-HS3.2-HS4)
(i) 3′ LTR (Delta-U3);
(j) SV40 poly (A) signal; and
(k) SV40 Ori.

Another object of the present invention is to provide recombinant lentivirus (LV) vector, wherein said viral vector encoded by nucleotide sequence of SEQ ID NO: 01 comprising:
(a) RSV Promoter;
(b) 5′ LTR;
(c) HIV-1 psi;
(d) RRE;
(e) cPPT/CTS;
(f) 3′ Beta Enhancer;
(g) HBB;
(h) Beta Globin Locus control Region (HS2-HS3-HS3.2-HS4)
(i) 3′ LTR (Delta-U3);
(j) SV40 poly (A) signal; and
(k) SV40 Ori.

Another object of the present invention is to provide recombinant cell, wherein said chimeric recombinant cell encoded by nucleotide sequence of SEQ ID NO: 01 comprising:
(a) 5′ LTR;
(b) HIV-1 psi;
(c) RRE;
(d) cPPT/CTS;
(e) 3′ Beta Enhancer;
(f) HBB (β-globin gene T88Q) with intrinsic promoter;
(g) Beta Globin Locus control Region (HS2-HS3-HS3.2-HS4) and
(h) 3′ LTR (Delta-U3).

Another embodiment of the present invention is to provide persistent expression of β-globin gene by using cell and/or gene therapy based administration of nucleotide sequence encoding β-globin gene for the treatment of thalassemia and sickle cell anemia.

Another embodiment of the present invention is to provide therapies to treat thalassemia using hematopoietic progenitor cells or mammalian stem cells transduced with lentivirus vector containing nucleic acid transgene encoding functional β-globin gene.

Another embodiment of the present invention is to provide methods for recombinant lentivirus (LV) particles preparation wherein, the lentivirus (LV) particle is packed with genes expressing functional β-globin gene in erythroid cell lineage specifically.

In one embodiments locus control region of β-globin gene (β-LCR sequences) that drives transcription of β-globin mRNA in erythroid lineage of the cells only. Where in large portions of the β-globin locus control regions which include large portions of DNase 1 hypersensitive sites HS2, HS3 (HS3 and/or HS3.2) and HS4. The regions may be the complete size or truncated one which provides the functionality of erythroid lineage specific regulation of β-globin expression.

In certain embodiments the vector genomes described herein include a polyadenylation signal (polyA). A variety of suitable polyA are known. In one example, the polyA is SV40 polyA signal. Still other suitable polyA sequences may be selected.

The embodiments of the present invention are further described using specific examples herein after. The examples are provided for better understanding of certain embodiments of the invention and not, in any manner, to limit the scope thereof. Possible modifications and equivalents apparent to those skilled in the art using the teachings of the present description and the general art in the field of the invention shall also form the part of this specification and are intended to be included within the scope of it.

### EXPERIMENTAL DETAILS

In the present invention, we evaluated the expression of therapeutic βA (T88Q)-globin gene derivative in CD34+ hematopoietic stem cells using lentiviral vector (LV) to develop futuristic, indigenous, cost effective, ex vivo genetically modified autologous CD34+ hematopoietic stem cell therapy product for the treatment of transfusion dependent β-thalassemia (TDT) and severe sickle cell disease (SCD).

### Example 1: Plasmid Vector Designing

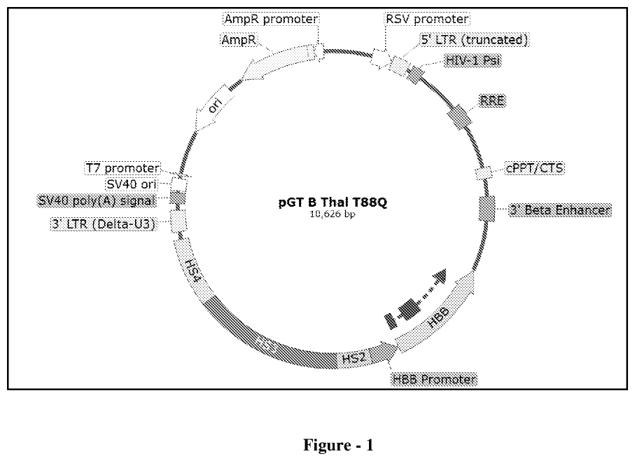

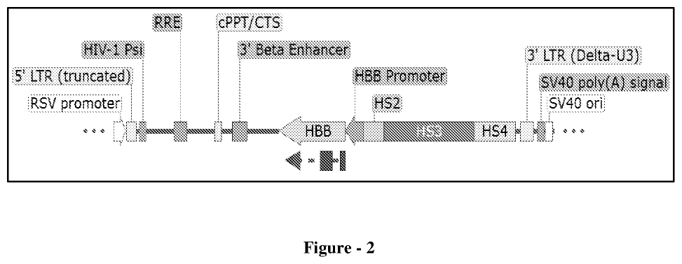

We designed a self-inactivating lentiviral vector pGT-BThal-T88Q encoding therapeutic β^{A} (T88Q)-globin gene derivative (human, adult, β^{A}-globin gene with a glutamine amino acid residue substituted for a threonine at position 88) under the control of native β-globin promoter, 2.7 kb of DNase I hypersensitive sites HS2, HS3, and HS4 of the human β-globin Locus Control Region (LCR), 3′ β-globin enhancer along with the intron-1 and intron-2 (372 bp IVS2 deletion in intron 2) as these elements control tissue specificity, developmental switching and confer high, erythroid-specific β-globin gene expression (FIG. 1 and FIG. 2). The construct was synthesized from GenScript USA, sequence confirmed and scaled up in-house.

### Example 2: Transformation, Banking and Bulk DNA Preparation

### Transformation, Clone Selection and Banking

The plasmid DNA was transformed in an E. coli strain stbl3. Transformants were scored over LB-kanamycin. Plasmid DNAs were isolated from the transformants using mimiprep and restriction digestion analysis was performed to characterize the plasmid for the presence of gene of interest. Among several correct transformant, a high performing (high purity, yield as well as high content of covalently closed circular plasmid) clone was selected and final glycerol banks were prepared by transforming either E. coli stbl3 strain.

### Large Scale DNA Preparation

A vial from the glycerol stock was grown over night at 37° C. for 16-18 hours in LB medium containing 50 μg/ml kanamycin. Bacterial pellet was subsequently harvested by pelleting the biomass via centrifugation at 4° C. and large scale plasmid preparation was performed using maxiprep or gigprep as per manufacturer's recommendation. Briefly, bacterial pellets were re-suspended in re-suspension buffer using a pipette followed by alkaline lysis, neutralization and column purification.

### Example 3: Generation of Transgene Packaged LV Particles and Titer Determination

LV particles were produced via either single plasmid (transfer plasmid carrying β-globin gene) transfection in LV producer cell line or four plasmid transient transfection (three helper plasmid and one transfer plasmid) in HEK293 cell variants using Lipofectamine 3000 or PEI-Pro/PEI-Max.

### Four Plasmid Transient Transfection in Adherent HEK293 Derived Cell Line:

HEK293FT cells were transfected with 4 plasmids in OptiMEM using lipofectamine 3000 transfection agent as per manufacturer's instruction. 24 hours post transfection the media was changed with cell line specific complete media. LV was harvested 48 and 72 hours after transfection, pooled, and clarified by low speed centrifugation followed by 0.45 μm filtration. The LV particles were concentrated (100×) using ultracentrifugation over sucrose density gradient and stored in aliquots at −80° C. Lentiviral titer determinations were done using p24 ELISA and titers were reported to be in the range of 8×10⁷ to 2×10⁸ transduction unit (TU)/ml.

### Single Plasmid Transient Transfection in HEK293 Derived Producer Cell Line:

To generate the LV in the HEK293 derived producer cell line i.e., PacLV, cells were propagated and maintained in 1000 mL shake flask in a suitable mammalian cell culture media. An exemplary option of such kind of media can be Hycell medium. Generally, the cells were seeded at 0.5 million/mL in 1000 mL shake flask with 300 mL media and maintained at 37° C., 100 rpm and 5% CO2 for 72 hours or until the cell density reached between 2.5-3.5 million/mL. A single transfection of producer cells was done with transfer plasmid (pGT-BThal-T88Q). The transfection of DNA was done using PEI-Pro and 3 hours post transfection, culture was induced by addition 10 nM of coumermycin and 80 ug/ml cumate. To enhance the LV production, 7 mM sodium butyrate and 2% v/v anticlumping agent were added 16 hours post transfection. Post 72 hours of transfection, culture was harvested, filtered and stored in −80° C. freezer till the purification. Lentiviral titer determination was done using p24 ELISA and titers were reported to be ˜7×10⁶ (TU)/ml.

### Example 4: Cell Line Maintenance

Different cell lines (i.e., K562 and HEK293 derivative cells) were propagated in their preferred media. Similarly, erythroid specific expression and production of functional β^{A} (T88Q)-globin gene derivative was tested in immortalized human erythroid progenitor (HUDEP-2) cell line.

### Example 5: In Vitro Evaluation for Functionality of Lentiviral Vector by Transduction and Determining Expression of β-Globin Gene

### The Expression of β^{A} (T88Q)-Globin Gene Derivative on Cell Lines:

The expression of β^{A} (T88Q)-globin gene derivative was tested in K562, a human erythroleukemic cell line and in HUDEP-2 cells after erythroid lineage specific differentiation. K562 cells and HUDEP-2 cells was transduced using β^{A} (T88Q)-globin LVs. Transduction efficiency was determined by flow cytometry and vector copy number (VCN) analysis.

Transduction and β^{A} (T88Q)-Globin Expression Analysis in CD34+ HSCs from Healthy Donors and Disease Subjects:

Functionally active β^{A} (T88Q)-globin production potential of β^{A} (T88Q)-globin gene derivative was studied in CD34+ HSCs isolated from healthy donor and/or disease subjects.

### Example 6: In Vivo Studies

In vivo POC studies were conducted to demonstrate the therapeutic potential of β^{A} (T88Q)-globin gene derivative in β-thalassemic mouse bone marrow cells transduced with β^{A}(T88Q)-globin LVs, or human healthy donor and/or haemoglobinopathy patients' CD34+ HSCs transduced with βA (T88Q)-globin LVs, administered to β-thalassaemic mice and immunodeficient NOD (NSG) mice respectively.

In POC studies, we evaluated successful and equivalent bone marrow engraftment, βA (T88Q)-globin production in long term engrafted bone marrow cells, any differences in bone marrow (BM) cellular differentiation, correction of dyserythropoiesis, correction of β-thalassemic phenotype, and any adverse effects associated on long-term bone marrow engraftment.

We claim:

1. A lentivirus (LV) vector comprising nucleotide sequence encoding β-globin gene.

2. The lentivirus (LV) vector according to claim 1, wherein β-globin gene comprises threonine to glutamine substitution at amino acid position 88 (T88Q).

3. A viral genome of recombinant lentivirus (LV) particle encoded by nucleotide sequence of SEQ ID NO: 01 comprising:
(a) One or more left and right long terminal repeat (LTR) sequences in its native or truncated form, that flank the 5′ or 3′ terminus of the heterologous polynucleotide sequence;
(b) HIV1 psi;
(c) RRE (Rev response element of HIV-1);
(d) An expression control elements; and
(e) SV40 PolyA Signal.

4. The viral genome of recombinant lentivirus (LV) particle according to claim 3, wherein the expression control elements comprises human β-globin promoter, a human β-globin LCR and a human β-globin enhancer sequence.

5. A recombinant lentivirus (LV) vector, wherein said viral vector comprising:
(a) RSV Promoter;
(b) 5′ LTR;
(c) HIV-1 psi;
(d) RRE;
(e) cPPT/CTS;
(f) 3′ Beta Enhancer;
(g) HBB (β-globin gene T88Q);
(h) Beta Globin Locus control Region (HS2-HS3-HS3.2-HS4)
(i) 3′ LTR (Delta-U3);
(j) SV40 poly (A) signal; and
(k) SV40 Ori.

6. The recombinant lentivirus (LV) vector according to claim 5, wherein said lentivirus vector is encoded by nucleotide sequence of SEQ ID NO: 01.

7. The recombinant lentivirus (LV) vector according to claim 5, wherein said lentivirus vector is used in gene therapy based administration of nucleotide sequence encoding beta globin gene for the treatment of thalassemia and sickle cell anaemia.

8. The recombinant lentivirus (LV) vector according to claim 5, wherein said lentivirus vector is used in cell therapy based administration of nucleotide sequence encoding beta globin gene for the treatment of thalassemia and sickle cell anaemia.
